(19) United States
(12) Patent Application Publication
(10) Pub. No.: US 2009/0098188 A1
(43) Pub. Date: Apr. 16, 2009

(54) Method for delivery of medication using a dissolvable device

(76) Inventor: Robert J. Staab, Old Lyme, CT (US)

Correspondence Address: EVELYN M. SOMMER, 570 LEXINGTON AVENUE , 17TH FLOOR, NEW YORK, NY, 10022, US

(21) Appl. No.: 12/287,253

(22) Filed: Oct. 8, 2008

Related U.S. Application Data
(60) Provisional application No. 60/998,383, filed on Oct. 11, 2007.

(51) Int. Cl. A61F 6/06; A61K 31/375

(52) U.S. Cl. 424/430; 514/474

(57) ABSTRACT

A method utilizing a dissolvable device for the internal delivery of medication and more particularly to the use of films made of a safe polymer material incorporating a medication that is released by dissolution of the film over time, and more particularly a treatment method for controlling or regulating the pH in the vagina by stabilizing and adjusting the pH in the vagina by minimizing the impact of the vaginal flora.

### FIELD OF THE INVENTION

The invention generally relates to a method utilizing a dissolvable device for the internal delivery of medication and more particularly to the use of films made of a safe polymer material incorporating a medication that is released by dissolution of the film over time. Still, more particularly the present invention relates to a treatment method for controlling or regulating the pH in the vagina by stabilizing and adjusting the pH in the vagina by minimizing the impact of the vaginal flora.

The method of the invention comprises applying a device adapted for local administration of an agent material into the vagina. The device comprises a dissolvable element and an agent material carried in such dissolvable element. During use, the heat and humidity in the vagina dissolves the dissolvable element and releases the agent. The dissolution properties of the dissolvable element can be readily tuned and controlled for rapid dissolution (for example in 5-60 seconds) or for dissolution over prolonged periods (for example up 12-24 hours), at least partially, by adding nitrogen or other suitable gas in forming a film of the dissolvable element.

### BACKGROUND ART

Due to the growing awareness of medical complications associated with imbalance of vaginal microflora and/or its related effect on pH, it is often desirable to administer medication into the vagina and that the medication be applied throughout the area of the vaginal tract and cervix over an extended period of time, for example several hours or days. The remoter areas of the vaginal tract might not be readily reached by conventional vaginal suppositories due to the compact size and shape required for convenience of insertion. Also, because of the structure and shape of the vagina, inserted suppositories or tablets often do not stay in place, or upon melting, the medication may drain out of the vaginal passage thereby reducing the effectiveness of the applied medication. Medicated tampons also do not extend far enough or widely enough into the vaginal tract to deliver medication throughout the vaginal tract. Other types of rigid applicators have similar problems and are uncomfortable to insert and use. Thus, under current methods, the desired medication may not be applied or maintained effectively in the vaginal tract for a sufficient period of time. More importantly, the known products suffer from deficiencies of all like prior art products in that they do not dissolve readily and in addition are not stable on prolonged storage at high temperature and high humidity, such as is generally encountered in numerous tropical countries as well as seasonally in more temperate climates. Such products become, under exposure to adverse humidity conditions, sticky and excessively hygroscopic. To resolve this problem, prior art devices, employ expensive protective packaging such as foil packs which greatly increases the cost of the product to the end user. The high cost discourages their use particularly in areas of the world where the product is most needed. Moreover, foil packaging increases package components and since the foils used are not readily decomposable in landfills such packages have a disadvantageous environmental impact.

It should be noted that as used herein, high temperature means up to 140° F., high humidity means up to 99% relative humidity and prolonged storage means in excess of three years. The present inventor has previously been granted U.S. Pat. Nos. 5,529,782 and 5,393,528 on devices adapted for local administration of an agent material in an internal body cavity. The entireties of which are incorporated herein by reference thereto.

It has occurred to the present inventor that the widespread condition of pH imbalance in the vagina and the changes in the microflora could be treated by application of a dissolvable element with adjustable dissolution properties and which carries at least one kind of desired agent material into the vaginal cavity. The dissolvable element remains in substantially solid form before use and dissolves primarily due to human body temperature and moisture during use to release the agent material in a desired time release and dosage.

### SUMMARY OF THE INVENTION

In accordance with the present invention, there is provided a method of controlling and adjusting the pH of the vaginal content by utilizing a device made of a safe polymer material incorporating a medication that is released by dissolution of the film over time. The device is adapted for local administration of an agent material in an internal body area such as the vagina and comprises a dissolvable element and an agent material carried in said dissolvable element wherein said dissolvable element is made of dissolvable polymer material, such as, polyvinyl alcohol, polyethylene oxide, and/or complex carbohydrate material, which is selected such that the dissolvable element remains in solid form before use, and dissolves due to human body temperature and moisture during use to release said agent material for local administration in the internal body area. The preferred dissolvable element is a film made of polyvinyl alcohol, polyethylene oxide, and/or a complex carbohydrate material such as hydroxypropyl methyl cellulose which are safe, food-grade materials selected to obtain a desired release characteristic for the agent material. Two or more film layers may be combined as a laminate for compound release properties. The dissolvable element dissolves within the body area so that it does not have to be physically removed after use. It can also dissolve completely when flushed away, so that no plumbing blockage or ecologically disturbing solid waste occurs.

The dissolution properties and texture of the dissolvable element may be modified by adding nitrogen or other suitable gases in forming the film, as well as the use of polyethylene oxide alone or in mixtures with polyvinyl alcohol and/or complex carbohydrate material. Forming the film of the invention with different film layers or polymer materials allows varied dissolution properties. The polyethylene oxide and complex carbohydrate materials add lubricity to the product as an added benefit. The composition of the dissolvable element is selected to have an improved heat and humidity stability, feel, texture, and dissolution time.

The active agent may be incorporated into either the entire portions of the device, i.e., as a homogeneous blend or in the case of a laminate, the device may include multiple layers, at least one layer containing one or more types of active materials. The dissolvable element may also be used to deliver contraceptives or medications such as anti-infectives, antiinflammatories, coronary vasodilators, anesthetics, antitussives, expectorants, estrogenic, progestational or prostaglandin agents, a homeopathic drug and the like, and combinations thereof. It may include fragrance, flavorants, coloring agents, preservatives, etc., to provide a more acceptable, environmentally sound product for consumers, as well as a plasticizer or gas additive for better handling, lubricity, and/or release characteristics. The film or the laminate of the films are preferably rolled into a cylindrically-shaped nonwoven device. The cylindrically-shaped device can be digitally inserted into body cavity (vagina) during use as a digital tampon. In an alternative embodiment, the cylindrically-shaped nonwoven device is encased in an applicator of the type conventionally used with tampons and the device can be delivered in the same manner as a tampon with an applicator.

### DESCRIPTION OF PREFERRED EMBODIMENTS

In the invention the treatment for normalizing the vaginal flora by controlling or adjusting the pH of the vaginal cavity comprises inserting into the vaginal tract a dissolvable device for delivery of medication which device contains a dissolvable element made of a dissolvable polymer material, particularly, a mixture of polyvinyl alcohol, polyethylene oxide, and/or complex carbohydrate material, used for local administration of a pH adjusting or normalizing agent possibly with another medication agent, into an internal body area. The dissolvable polymer material is preferably a food-grade material safe for internal use. The dissolvable element is designed to be heat stable (e.g., up to 140° F.), and humidity stable (e.g., up to 99% relative humidity) so as to remain in substantially solid form and not begin dissolving before its intended use. Lubricity is another desirable property for use in the vagina where sensitive tissues are likely to be encountered.

The preferred dissolvable element is in the form of a film made of the combination of grades of polyvinyl alcohol, polyethylene oxide, and/or complex carbohydrate material. Polyvinyl alcohol (PVA) is a preferred material for the film because it is non-toxic and medically safe to use internally. PVA comes in different grades that can be classified as cold water soluble (dissolves from 40° to 212° F.), intermediate dissolving (110° to 212° F.), fully hydrolyzed (140° to 212° F.), and superhydrolyzed (180° to 212° F.) PVA is commercially available from companies such as Air Products Company, of Allentown, PA. The cold water soluble and intermediate dissolving grades are the most useful for the desired moisture and heat dissolving properties for contraceptive grade purposes. A particularly preferred cold water soluble grade of PVA is an 80% hydrolyzed polyvinyl alcohol having a molecular weight of 9,000-10,000; for intermediate solubility, an 87-89% hydrolyzed polyvinyl alcohol having a molecular weight of 13,000-23,000 for a slow dissolving, a 98-99% hydrolyzed polyvinyl alcohol having a molecular weight of 31,000 to 50,000 and for the least dissolving, a fully hydrolyzed >99% of polyvinyl alcohol having a molecular weight of 85,000-186,000 being preferred. All of the aforementioned polyvinyl alcohol preparations are available from Aldrich Chemical, Milwaukee, Wis. However, in the invention, a film of the higher temperature or water-soluble grade may be combined with a film of the lower temperature dissolution and moisture, solubility and stability properties so that the film can be used most suitably in the vaginal environment. The PVA material or materials are selected to dissolve relatively quickly, e.g., over several minutes, or in some cases as low as several seconds but may be selected for a longer release time, such as several days.

Polyethylene oxide is another good material for the film because it has very good moisture, particularly humidity, stability and further is a food contact grade material. It is very compatible with the pH modifying agents such as ascorbic acid and lactic acid and many other medications. It also has the added benefit of good lubricity, which makes the film structure even more comfortable to insert and use. Preferred polyethylene oxide materials are sold by Aldrich Chemical of Milwaukee, Wis., in molecular weights of from 50,000 to 8,000,000 Daltons.

The use of inert gases such as nitrogen, in forming the film to modify the dissolution properties of the dissolvable element formed from polyethylene oxide has been found to be equally favorable as their use in connection with the polyvinyl alcohol films. The dissolution of the film can be readily adjusted by using different viscosities of the hydroxypropyl methyl cellulose ranging from less than 80 to more than 4,000 centipoises.

A complex carbohydrate material suitable for use in the film is hydroxypropyl methyl cellulose, or carboxy methyl cellulose which is sold, for example, under the trademark “Methocel” by Dow Chemical of Midland, Michigan. This material is also food-grade, medically safe to use internally, low-cost, and very stable in a humid environment “Methocel” is cellulosic in nature being derived from trees. It is dissolvable in the same temperature ranges as PVA. Hydoxypropyl methyl cellulose is a particularly preferred material in the same temperature ranges as PVA. Its acceptance by the FDA as a direct food additive is well known (CAS 9004-65-3). The preferred hydroxypropyl methyl cellulose has an average molecular weight of about 86,000.

The film may be a laminate of two or more layers of different polymer materials, or may be a single layer with two or more polymer ingredients mixed together. Further, the film laminate may be a gas foamed film or constructed of layers of different gas foamed films or layers of both non-foamed and gas foamed films. The exact mixture used will depend upon the intended use and combination of qualities desired, which may include heat-dissolving dissolving temperature range, time release period, lubricity, shelf life, turgidity, stability in a moisture environment, compatibility with spermicides and/or medications etc. In the case of contraception, two films may be used which dissolve at varying rates. Such a laminate device can offer prompt efficacy upon insertion combined with extended contraceptive protection with dissolution taking place over a period of many hours. Thus in accordance with the invention, the film may be constructed as a laminate composed of gas foamed film with non-gas foamed film layers, polyvinyl alcohol and polyethylene oxide film layers, polyvinyl alcohol and hydroxypropyl methyl cellulose layers, in all possible combinations. The laminates can be formed in the conventional manner, for example the mixture in liquid form will be poured or cast on to a plate or into a mold and allowed to begin to set, at which time another liquid mixture of different composition will be poured onto the first setting up mixture, and both mixtures allowed to set up completely producing a laminate or layers of different materials.

Fully formed films can also be laminated to each other through use of an adhesive. A preferred adhesive is a dilute aqueous solution of the polymer from which the film has been made. Thus, for example, a polyvinyl alcohol film could be adhered to another polyvinyl alcohol film through use of a dilute solution of polyvinyl alcohol.

The agent material to be administered locally in particular the agent for stabilizing and in adjusting the pH in the vagina, may include drugs, contraceptives or medications. The agent material is evenly distributed throughout the film, so that as the film slowly dissolves, it releases the agent material in the proper dosage to perform its pH adjusting effect and other medicating function. If a laminate of films is employed, more than one layer of the laminate can contain actives of the same or different identities. The agent material is selected for compatibility with the polymer material and its dissolution characteristics. The device of the invention thus is composed of a biologically compatible material that has been blended homogeneously with a pH adjusting or controlling agent possibly a spermicide and/ or drug which is released into a body cavity at a controlled rate upon contact with the body fluid.

The dissolvable element may be used to deliver the pH control or the adjustment agent alone, internally in the vagina or cervical area or in combination with other suitable medications which can be delivered with the film and include: (1) anti-infectives such as antibiotics, sulfonamides, antivirals, antifungals, antiprotozoan and antibacterials; (2) anti-inflammatories, such as hydrocortisone, dexamethasone, triamcinolone, and various prednisolone compounds; (3) estrogenic steroids, such as estrone; (4) progestational agents, such as progesterone; (5) prostaglandins; (6) coronary vasodilators; (7) antitussives; (8) antihistamines; (9) anesthetics, and (10) homeopathic drugs. Monoclonal antibodies such as those useful against cell surface components or against pathogenic organisms such as the human-immuno-deficiency (HIV) family of viruses may be incorporated into the device of the present invention for ultimate intravaginal release. Combinations of the various drugs may be used as desired. Typically the range of drug additives may be in the amount of 0.0001% to about 50% by weight. The pH stabilizing and/or adjusting agents and other medications may be in a variety of chemical forms, such as uncharged molecules, molecular complexes, or non-irritating, pharmacologically acceptable salts. Simple derivatives of such medications, such as ethers, amides, and the like, can also be used for desirable properties such as retention, release, and easy hydrolyzation by body pH, enzymes, etc. The amount of medication to be used varies depending upon the condition, the particular drug, the desired therapeutic or prophylactic effect, and required release times. Examples of pH adjusting agents include ascorbic acid, vitamin C and the like. Other drugs include clotrimazole, miconazole, tiaconazole, benzalkonium chloride, nystatin, dermally active steroids, hormones, benzocaine, sulfas, biologically prepared actives, psychotropics, nitroglycerine, etc. If the drug can be applied on or in a moist area of the body, such as the mouth, skin, vagina, rectum, ear canal, eye, etc. then the film can be used to deliver the drug effectively with timed release of the proper dosage. This should be an ideal way for treating ulcers of the mucous membranes and of the skin for adjusting and/or stabilizing the pH of the vagina as well as for treating bum wounds.

The dissolvable element may also include a plasticizer material, such as water, glycols, glycerin and like materials in order to enhance lubricity and softness. While water is suitable as a plasticizer it is not useful in all cases, but this factor can be readily ascertained. A preferred plasticizer is glycerin USP, sold by Van Waters & Rogers, Inc., in either natural or synthetic form. Glycerine (glycerol) CAS 56-81-5 is particularly preferred.

The plasticizer may be added in any desired concentration, for example, from 0.1% to 35%, for better handling and lubricity. The softness and flexibility of the dissolvable film, due to its thin layer structure without any rigid elements, and particularly when combined with plasticizer, ensures that the device may be used with complete comfort.

Various preservatives, antifungal agents, antibacterial agents, antiviral agents, antiprotozoal agents, and antioxidants may also be added if desired. Flavors, fragrances, and/or coloring agents may also be added. The polymer film may be substantially transparent, or may be embossed with indicia or colored with opaquing agents. These additives may be present in any desired concentration, for example, from 0.001% to 50%. The concentrations of these additives will depend upon the condition to be treated, the agent's desired properties, the agent to be released, the potency, the desired dosage, dissolution times, etc.

In preparation, the polymer solids, water, or other solvent, medicinal, glycerine etc. are admixed in the proper concentrations and the mixture heated to the appropriate temperature for dissolution and formation of a uniform blend to take place. The heating can take place, for example, by submerging vessels containing the mixture in water or jacketed vessels held at constant temperature, for example 104°-140° F. The mixture can either be cast directly or transferred into another water bath of cooler temperature, for example 68°-104° F. and other heat sensitive ingredients introduced with stirring. The application of heat is, however, not necessary, which is advantageous when pharmaceuticals or other agents to be added are heat sensitive. Several formulations utilizing different polymers as well as different active ingredients are listed below:

### For Film Delivery of Ascorbic Acid Ph Controlling Agent

### EXAMPLE 1

| | | |
|---|---|---|
| Carboxypropyl methyl cellulose | 165 | mg |
| Glycerine | 20 | mg |
| Ascorbic acid | 45 | mg |

### EXAMPLE 2

| | | |
|---|---|---|
| Carboxypropyl methyl cellulose | 165 | mg |
| Ascorbic acid | 45 | mg |

### EXAMPLE 3

| | | |
|---|---|---|
| Polyvinyl alcohol | 165 | mg |
| Glycerine | 20 | mg |
| Ascorbic acid | 45 | mg |

### EXAMPLE 4

| | | |
|---|---|---|
| Polyvinyl alcohol | 165 | mg |
| Ascorbic acid | 45 | mg |

### Film for Delivery of Surfactant Spermicide

### EXAMPLE 5

| | | |
|---|---|---|
| Carboxypropyl methyl cellulose | 105 | mg |
| Glycerine | 18 | mg |
| Nonoxynol - 9 | 100 | mg |

### EXAMPLE 6

| | |
|---|---|
| Carboxypropyl methyl cellulose | 105 mg |
| Nonoxynol - 9 | 100 mg |

### EXAMPLE 7

| | | |
|---|---|---|
| Polyvinyl alcohol | 105 | mg |
| Glycerine | 18 | mg |
| Nonoxynol - 9 | 100 | mg |

### EXAMPLE 8

| | |
|---|---|
| Polyvinyl alcohol | 105 mg |
| Nonoxynol - 9 | 100 mg |

A second method of preparing the film involves the addition of the active by way of a final water bath. There are typically several baths during the manufacturing process. The final bath can contain one or more active for addition to the film to be formed.

A third method of preparing the film involves the inclusion of one or more actives in the polymeric pellets. The pellets of the polymers (such as polyvinyl alcohol)_can be manufactured containing one or more actives. When the polymer pellets are used for the production of the film, the actives are carried along with the pellets.

The film characteristics may also be altered by adding appropriate amounts of gas, such as air, nitrogen, or other inert gases, in the manufacturing process which can produce a more acceptable film texture and modify the dissolution rates accordingly. For example, it has been found that the addition of nitrogen or other inert gas to a PVA film containing nonoxynol-9 halves the dissolution rate of the film. The fine-tuning of dissolution rates and delivery of agent material, by the addition of gases and by altering the grades or mixtures of polymer materials or layers, is an important aspect of the invention.

On addition of the gas, preferably nitrogen, a web is formed of the final formulation and the gas. The resultant structure can be described as a foam with various sized air bubbles trapped in the matrix. There is a dual benefit that has been surprisingly observed in this connection, namely that not only can the size of the bubbles in the foam alter the dissolution rates and correct what is a serious flaw in standard polymer films, it also offers to the user a perceptible softness to the film which enables the delivery of many types of drugs to tender mucosal tissues. It has been observed that the formation of this web of the polymer/drug formulation with the gas must be made just prior to casting. This offers precise control over the microbubbles and resultant control over the dissolution.

With this web formation, the quick release of drug is made possible. This frothy foam mixture or web can also be added to a mold to provide a formed device such as a barrier delivery system which completely dissolves upon use in a body cavity, e.g. the vagina.

The gases, for example air or nitrogen are introduced near the point of application of the liquid polymer material to the stainless steel casting sheet. The gases are added in a closed system by mixing with whipping blades or a motor driven homogenizer to homogenize the mixture of polymer, active material and gas to form a frothy foam. The final mixture then sets up or gels as a foam.

The method of the invention utilizing the dissolvable film, for internal delivery of pH adjusting and/ or stabilizing agent medication in accordance with the present invention, has a number of advantages over use of conventional contraceptives and tampon and sponge applicators. It is fully dissolvable and environmentally safe for disposal, and does not require removal, cleaning refitting or replacement. It is made of natural, food grade materials for safe internal use, and can be manufactured free of any irritant or toxic chemicals. Tests show that the film can be made to dissolve to deliver the agent in less than half the time required for other film devices. It is very convenient and not messy to apply and use, and will not stain clothing as with creams and gels. It causes no irritation and can be readily inserted by hand and does not require an applicator. The film can also be used at the same time to administer desired medications in internal areas of the body.

While the film or the laminate of film can be used directly during application, a nonwoven device produced by rolling the film or laminate of film is preferred. The nonwoven rolled device can be manufactured, packaged, sold and used as a digital tampon for application without the aid of an applicator. Alternatively, the nonwoven rolled device can be encased in an applicator during manufacturing and can therefore be inserted with the aid of the applicator. The applicator can be the kind conventionally associated with tampons and made of plastic or paper. The nonwoven rolled device can further be compressed for enhanced capacity within the same volume.

Numerous variations are of course possible in the light of principles and examples disclosed above. All such variations are intended to be included within the entire spirit and scope of invention, as defined in the following claims.

I claim:

1. A method for the delivery of an agent material into the vagina for normalizing or controlling the pH in the vagina and normalizing the vaginal flora, using a device characterized by improved heat and humidity stability, said device comprising at least one dissolvable film formed from a member selected from one group consisting of polyvinyl alcohol, polyethylene oxide,
hydroxypropylmethyl cellulose and mixtures thereof, said agent material being incorporated in said dissolvable film, said device dissolving at the temperature of said vagina in the presence of the moisture naturally present therein to release said agent material, said method comprising introducing said device into said vagina, whereby on dissolution of said film said agent is delivered into the vagina.

2. A method according to claim 1 wherein said film is formed of a laminate comprising of at least two film layers, each characterized by different temperature dissolution properties.

3. A method according to claim 1 wherein said dissolvable film additionally contains a spermicide for contraceptive use and/or a medication.

4. A method according to claim 3 wherein said agent material is a spermicide, said device is adapted for use as a contraceptive and said method comprises inserting and positioning said device in the vagina.

5. A method according to claim 1 wherein said agent material is ascorbic acid.

6. A method according to claim 1 wherein the dissolution rate of said film is adjusted by introducing an inert gas into said film.

7. A method according to claim 4, wherein additionally contains a medication which is a member selected from the group consisting of anti-infectives, antiinflammatories, estrogenic steroids, progestational agents, prostaglandins, coronary vasodialators, antitussives, antihistamines, and anesthetics.

8. A method for topical for topical delivery of an agent material to an external body area using a device characterized by improved heat and humidity stability of up to 140° F. and up to 97% respectively, said device comprising at least one dissolvable film formed from a member selected from the group consisting of polyvinyl alcohol, polyethylene oxide, hydroxypropyl cellulose and mixtures thereof, at least one of said films having distributed throughout an inert gas, said agent material being incorporated in said dissolvable films and dissolution at the temperature of said body cavity in the presence of the moisture naturally present in said body cavity to release said agent material, said method comprising applying said device onto an external body area, whereby on dissolution of film said agent material is delivered topically to said external body area.

9. A method according to claim 1, wherein said film is rolled into a cylindrical body.

10. A method according to claim 9, wherein said cylindrical body is encased in an applicator prior to use.
